(19) United States
(12) Patent Application Publication
(10) Pub. No.: US 2013/0144281 A1
(43) Pub. Date: Jun. 6, 2013

(54) Tissue Treatment Apparatus with Interchangeable Instrument/Accessories

(71) Applicants: Reuven Lewinsky, Alonei Aba (IL); Roee Khen, Haifa (IL); Nissim Hay, Netanya (IL)

(72) Inventors: Reuven Lewinsky, Alonei Aba (IL); Roee Khen, Haifa (IL); Nissim Hay, Netanya (IL)

(73) Assignee: LUMENIS LTD, Yokneam (IL)

(21) Appl. No.: 13/699,669

(22) PCT Filed: May 24, 2011
(86) PCT No.: PCT/IB2011/001474

§ 371 Date: Feb. 8, 2013

Related U.S. Application Data
(60) Provisional application No. 61/348,694, filed on May 26, 2010.
(60) Provisional application No. 61/349,421, filed on May 28, 2010.

(51) Int. Cl. A61B 18/22

(52) U.S. Cl. CPC A61B 18/22
USPC 606/16

(57) ABSTRACT

A tissue treatment apparatus is provided for surgical and medical procedures and operations including an instrument and accessory base adapted for receiving one or more instruments and/or accessories configured for different procedures and operations and for a variety of modes of use of the treatment apparatus.

### FIELD OF THE INVENTION

This invention relates to the field of surgical instruments, and in particular to an instrument for laser treatment of a targeted tissue of a patient with the addition of a variety of treatment tips for performing specific functions.

### BACKGROUND OF THE INVENTION

Surgical instruments can be designed and constructed as rigid, articulated and/or flexible devices in order to achieve specific effects on target tissues, such as cutting, ablation or coagulation of tissue. In addition, surgical instruments can be designed and constructed as “cold” or mechanical devices that achieve one or more desired effects mechanically, or can be designed and constructed as “hot” devices that are energized by different energy sources, such as, for instance, radio frequency, ultrasound and laser energy sources, to manipulate/treat tissue. Other surgical instruments can be designed and constructed as both “cold” and “hot” devices that perform both cold and energized tissue manipulation/treatment.

An advancement of laser surgery includes a tissue-treating laser beam that can cut tissue at a distance from a laser energy delivery device without requiring direct contact between a laser treatment instrument and the target tissue. The distance between, for example, the tip of a laser instrument and the target tissue can be determined by a surgeon based on his experience and judgment during a surgical operation. This is one way a surgeon controls the power density applied to the target tissue. However, in other cases, it is preferred or required to maintain a specific distance between the target tissue and the laser instrument tip. In these cases, a standoff is typically used to help to attain and maintain a preferred or required distance between the laser instrument tip and the target tissue.

Other surgical procedures may require a backstop mechanism that prevents a laser beam from reaching non-target tissue beyond the intended tissue treatment site. Other surgical procedures also may require the ability to couple the laser beam to energetic effects of an ultrasonic energy or radio frequency energy or to some mechanical dissection apparatus.

Thus, it is desirable that a tissue treatment instrument be designed and constructed to receive or accept, and to interchange with, multiple instruments or accessories, with each instrument or accessory constructed and arranged for a specific procedure and/or for a particular mode of use of the treatment instrument. In addition, it is desirable that the tissue treatment instrument include an instrument and accessories tip or base that is adapted to permit the instrument to receive or accept, and to interchange with, multiple instruments or accessories. Such a treatment instrument and accessories base permits a surgeon or operator to couple any of multiple instruments or accessories to the instrument, such as, for example, for a specific procedure or mode of use of the instrument. Such an instrument and accessories base facilitates flexibility in terms of various procedures that the instrument may perform, various modes of use of the instrument, as well as various instruments or accessories that the instrument can receive or accept. Such instruments or accessories may include, but are not limited to, any of a variety of standoffs, backstops, dissecting devices, aiming devices, and other procedure- or mode of use-specific elements.

### SUMMARY OF THE INVENTION

In one aspect, an apparatus for treating a patient's tissues is disclosed.

In another aspect, the apparatus includes a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip is a standoff mounted on the distal end of the accessories base that extends a distance from the distal end and terminates a selected distance from the distal end.

In another aspect, the apparatus further comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip is a backstop mounted on the distal end of the accessories base that extends a distance from the distal end and terminates a selected distance from the distal end.

In yet another aspect, the apparatus comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip is a beam deflector mounted on the distal end of the accessories base, the beam deflector having a passage for receiving the flexible waveguide and bending the waveguide a selected amount from the longitudinal axis of the flexible waveguide.

In still another aspect, the apparatus further comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip is a beam deflector mounted on the distal end of the accessories base, the beam deflector comprising a housing having a reflector disposed at an angle with respect to the beam of laser radiation from the flexible waveguide to deflect the beam of laser radiation impinging on the reflector and redirect the beam to a target tissue of a patient.

In another aspect, the reflector is disposed at an angle to deflect the beam approximately 90°.

In another aspect, the reflector is disposed at an angle to deflect the beam approximately in the range of 1° to 90°. The reflector may be a mirror.

In another aspect, the apparatus of claim 4 further comprises a conduit associated with the support structure, the conduit being capable of directing a fluid at the reflector to clear debris from the reflector.

In another aspect, the apparatus further comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip is a dissector mounted on the distal end of the accessories base, the dissector enclosing the distal end of the flexible waveguide, the waveguide being located spaced back from the distal end of the treatment tip to substantially eliminate contact of the distal end of the flexible waveguide and the target tissue of a patient.

Further, the apparatus comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip comprises at least a two lumens mounted on the distal end of the accessories base, one of the at least two lumens containing the flexible waveguide, the other of the at least two lumens containing one of more of: an optical fiber, a source of fluid, a temperature sensoring device, an illumination optical fiber, a vision sensing device, and a tissue analyzer.

In a further aspect, the apparatus comprises a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient; wherein the treatment tip is mounted at its proximal end on one of a removably mounted or permanently mounted accessories base on the distal end of the support structures; and wherein the treatment tip further includes at least one further mounting, the at least one further mounting being adapted to support at least one of: a standoff, a j-hook, a backstop, a beam deflector, a beam deflector with reflector, a dissecting device, an illuminating device, a sensor device, a viewing device, and a fluid carrying device.

Details of one or more implementations are set forth in the accompanying drawings and in the descriptions below. Further features, aspects and advantages will become apparent from the descriptions, the drawings and the claims.

### BRIEF DESCRIPTION OF THE DRAWINGS

FIG. 1 is a schematic diagram of a tissue treatment instrument including an accessories base according to one aspect of the invention;

FIG. 2 is a schematic diagram of the instrument shown in FIG. 1 including a standoff device mounted or connected to the accessories base;

FIG. 3 is a schematic diagram of the instrument shown in FIG. 1 including a backstop device mounted or connected to the accessories base;

FIG. 4 is a schematic diagram of the instrument shown in FIG. 1 including a beam deflector mounted or connected to the accessories base;

FIG. 5 is a schematic diagram of the instrument shown in FIG. 1 including a beam deflector having a mirror mounted or connected to the accessories base;

FIG. 6 is a schematic diagram of the instrument shown in FIG. 1 including a dissection device mounted or connected to the accessories base;

FIG. 7 is a schematic diagram of the instrument shown in FIG. 1 including a multi-channel tip device; and

FIG. 8 is a schematic diagram of a tissue treatment instrument including a wave guide or optic fiber holder according to another aspect of the invention.

### DETAILED DESCRIPTION OF THE INVENTION

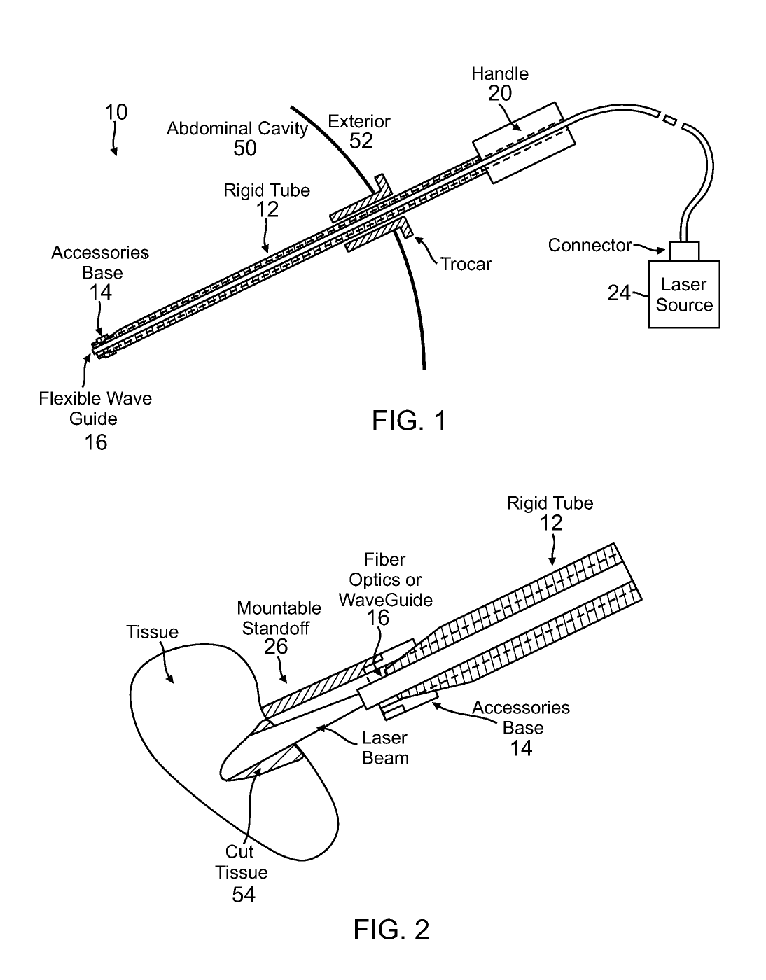

Referring to FIG. 1, in one aspect, the invention provides a tissue treatment instrument 10 constructed and arranged for surgical and medical procedures and operations. In one embodiment, the tissue treatment instrument 10 is a laser instrument including a rigid tube 12 operatively connected to a laser source 24 and constructed and arranged to receive a waveguide 16 for delivery of a laser beam to target tissue. The rigid tube 12 includes at its distal end or tip an accessories base 14 constructed and arranged to receive or accept one or more instruments or accessories according to the invention that are constructed and arranged for any of a variety of surgical and medical procedures and operations, as well as for any of a variety of modes of use. In one embodiment, the accessories base 14 is designed and configured as a separate mountable/connectable unit for mounting/connecting to the instrument 10, e.g., per a user's need. In another embodiment of the accessories base 14, the accessories base 14 is designed and configured integral with the instrument 10 such that the accessories base 14 and the instrument 10 are one unit onto which various instruments or accessories may be mounted/connected.

In another embodiment according to the invention, an endoscope replaces the treatment instrument 10. The wave guide 16 is received by a working channel defined within the interior of the endoscope and passes through the channel and the distal end of the endoscope. In this embodiment, the accessories base 14 is designed and configured as a mountable/connectable unit that is mounted/connected to the endoscope distal end.

In a further embodiment according to the invention, the instrument 10 may be constructed and arranged for procedures and operations including, but not limited to, ENT (ear, nose, and/or throat), gynecological, and neurosurgical procedures and operations, and may be constructed and arranged to provide, in addition to the laser energy delivery, other types of energy, such as radio frequency and/or ultrasound energy.

In other embodiments according to the invention, the instrument 10 may facilitate various methods of delivery of treatment and may be constructed and arranged as a rigid, articulated and/or flexible instrument 10 to achieve such treatment methods. The instrument 10 may also be a part of a wholly or partially articulated robotic instrument.

Regardless of the type of tissue treatment instrument 10, the accessories base 14 according to the invention is constructed and arranged to receive or accept a variety of instruments or accessories. The accessories base 14 and the instruments or accessories, as described in detail below, are constructed and arranged to permit connection and detachment of the instruments or accessories to the base 14 and to enable an interchange of instruments or accessories, such as, for example, during a procedure or an operation. The flexibility that the accessories base 14 and the instruments or accessories provide help to enable quick and relatively easy attachment and removal of the instruments or accessories to and from the accessories base 14 to thereby enable a quick change or alteration of instruments or accessories of the instrument 10 according to a preferred or required action on target tissue.

The invention anticipates that the instrument 10 according to the invention and, in particular, the accessories base 14 and the multiple accessories or instruments the base 14 accepts or receives according to the invention, are not limited and may include a variety of instruments and accessories or instruments for use in surgical, medical and other procedures and operations. The invention is disclosed below in connection with a laser laparoscope for illustrative purposes only and is not intended to limit the scope and spirit of the invention.

Still referring to FIG. 1, the laser instrument 10 (shown as a laparoscopic instrument in FIG. 1 only for the purposes of illustration) includes a rigid tube 12 defined with an elongate hollow member that is operatively connected to a handle 20. A flexible waveguide 16 is operatively coupled into and through the tube 12 and is maintained in place relative to the tube 12 and the handle 20. The wave guide 16 is operatively connected at its proximal end to the laser source 24. The tube 12 is configured to permit its insertion into, for example, an abdominal cavity 50, e.g., via a trocar 22, through a surgical incision in the exterior 52 of the abdominal cavity.

The distal end or tip of the tube 12 includes the accessories base 14 according to the invention. The accessories base 14 may be mounted or connected, e.g., removably, to the distal tip of the tube 12, or the accessories base 14 may be an integral part of the distal end or tip. The accessories base is constructed and arranged to receive or to accept one or more instruments or accessories according to the invention. The accessories base 14 thereby enables attachment or removal of multiple instruments or accessories to the instrument 10, such that, the instrument 10 may be used in one or more surgical, medical or other procedures, and/or the instrument 10 may be used in one or more modes of use.

The accessories base 14 is designed and configured with any of a variety of mounting or connection devices and configurations that facilitate mounting or connecting of an instrument or accessory to the accessories base 14. Any particular mounting or connection device and configuration of the accessories base 14 complements, or is similar to, a mounting or connection device and configuration of an instrument or accessory for use with the instrument 10. Such mounting or connection devices and configurations can include, but are not limited to, complementary threads along the accessories base 14 and instrument or accessory (for screw-type connections), snap connectors, Luer lock connectors typically used in surgical equipment, and other suitable connectors. In particular, such mounting or connection devices and configurations are suitable for permitting easy and quick connection and removal of an instrument or accessory to and from the accessories base 14.

In this manner, the accessories base 14 readily and easily receives or accepts and thereby mounts or connects an instrument or accessory to the instrument 10. In one embodiment of the invention, the accessories base 14 includes a mounting or connection device and configuration that are universal with respect to a multiple of instruments or accessories, such that, the accessories base 14 receives or accepts and thereby mounts or connects any of the multiple of instruments or accessories to the same instrument 10. The instrument 10 is thereby flexible in terms of the modes of use of the instrument 10, such as, for example, during a specific procedure/operation and in terms of the variety of procedures/operations in which the instrument 10 may be used. Specific instruments and accessories according to the invention are described in detail below with reference to FIGS. 2-7. The invention, however, is not limited to the instruments and accessories that are disclosed below and envisions that any of a variety of instruments and accessories may be used with the accessories base 14.

Referring to FIG. 2, in one embodiment of the invention, an instrument or accessory according to the invention includes a standoff or spatula 26 that is designed and configured to removably mount or connect to the accessories base 14. As mentioned above, in one embodiment of the accessories base 14 according to the invention, the accessories base 14 is designed and configured as a separate mountable/connectable unit for mounting/connecting to the instrument 10. In another embodiment of the accessories base 14 according to the invention, the accessories base 14 is designed and configured integral with the instrument 10 such that the accessories base 14 and the instrument 10 are one unit onto which various instruments or accessories may be mounted/connected. The standoff 26 may be constructed and arranged with a mounting or connection device and configuration for mounting/connecting to and removing from the accessories base 14. The standoff 26 may also be constructed and arranged to control the distance between the wave guide 16, which delivers the laser beam, and target tissue 54 once the standoff 26 is connected to the accessories base 14. Maintaining a specific distance between the wave guide 16 and the target tissue 54 helps to prevent or at least minimize accidental or inadvertent contact between the wave guide 16 and the target tissue 54. In addition, maintaining a specific distance helps to prevent or at least minimize over exposure of the target tissue 54 to laser energy since the laser beam can cause high power densities at short working distances. The standoff 26 also helps a surgeon to better estimate the expected location on the target tissue 54 where a laser beam emitted from the wave guide 16 will hit. The standoff 26 is more critical in the absence of an aiming beam. The standoff 26 is further designed and configured with such a profile size that it will not interfere in passing the instrument 10 through a trocar 22 or other device that enables such passage when the standoff 26 is mounted on the accessories base 14.

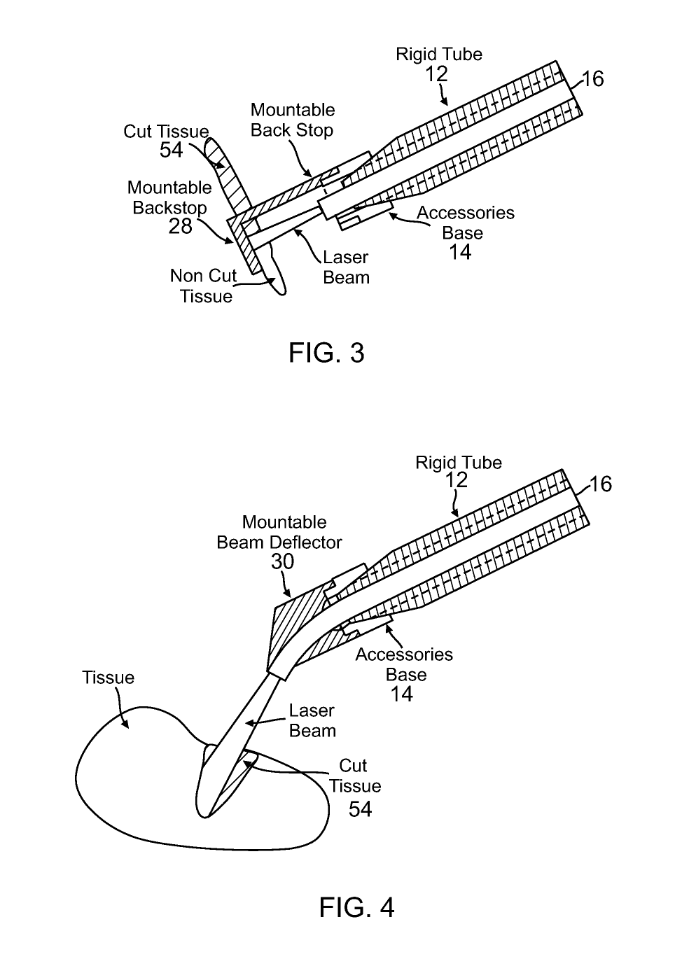

Referring to FIG. 3, in another embodiment of the invention, an instrument or accessory according to the invention includes a backstop 28 that is designed and configured to removably mount or connect to the accessories base 14. In one embodiment of the accessories base 14 according to the invention, the accessories base 14 is designed and configured as a separate mountable/connectable unit for mounting/connecting to the instrument 10. In another embodiment of the accessories base 14 according to the invention, the accessories base 14 is designed and configured integral with the instrument 10 such that the accessories base 14 and the instrument 10 are one unit onto which various instruments or accessories may be mounted/connected. The backstop 28 may be used with target tissue 54 that is relatively thin and where damage behind the target tissue 54 needs to be avoided or at least minimized. The backstop 28 is designed and configured to absorb excessive energy that is not absorbed by or passes through the target tissue 54. As shown in FIG. 3, once attached to the accessories base 14, the backstop 28 is positioned to help to prevent or to at least minimize damage behind the target tissue 54. In addition to its function as a back stop, the backstop 28 also helps to determine the cutting distance similar to that ability provided by the standoff 26. One of ordinary skill in the art would appreciate that a variety of backstops 28 of different sizes, thicknesses and materials may be employed with the accessories base 14 as may be dictated by the required application/procedure, organ geometry, beam spot sizes and other parameters of the procedure and/or the target tissue 54, such that a surgeon may select the most appropriate backstop 28 for use with the instrument 10. The backstop 28 is further designed and configured with a profile and size such that it will not interfere in passing the instrument 10 through a trocar 22 or other device that enables such passage when the backstop 28 is mounted on the accessories base 14.

Referring to FIG. 4, in a further embodiment of the invention, an instrument or accessory according to the invention includes a beam deflector 30 that is designed and configured to removably mount or connect to the accessories base 14. In one embodiment of the accessories base 14 according to the invention, the accessories base 14 is designed and configured as a separate mountable/connectable unit for mounting/connecting to the instrument 10. In another embodiment of the accessories base 14 according to the invention, the accessories base 14 is designed and configured integral with the instrument 10 such that the accessories base 14 and the instrument 10 are one unit onto which various instruments or accessories may be mounted/connected. The beam deflector 30 is constructed and arranged with a mounting or connection device and configuration for mounting/connecting to and removing from the accessories base 14. The beam deflector 30 is also constructed and arranged to facilitate bending of a flexible wave guide 16 to any of a preferred or required angle. The beam deflector 30 is further designed and configured with a profile and size such that it will not interfere in passing the instrument 10 through a trocar 22 or other device that enables such passage when the beam deflector 30 is mounted on the accessories base 14.

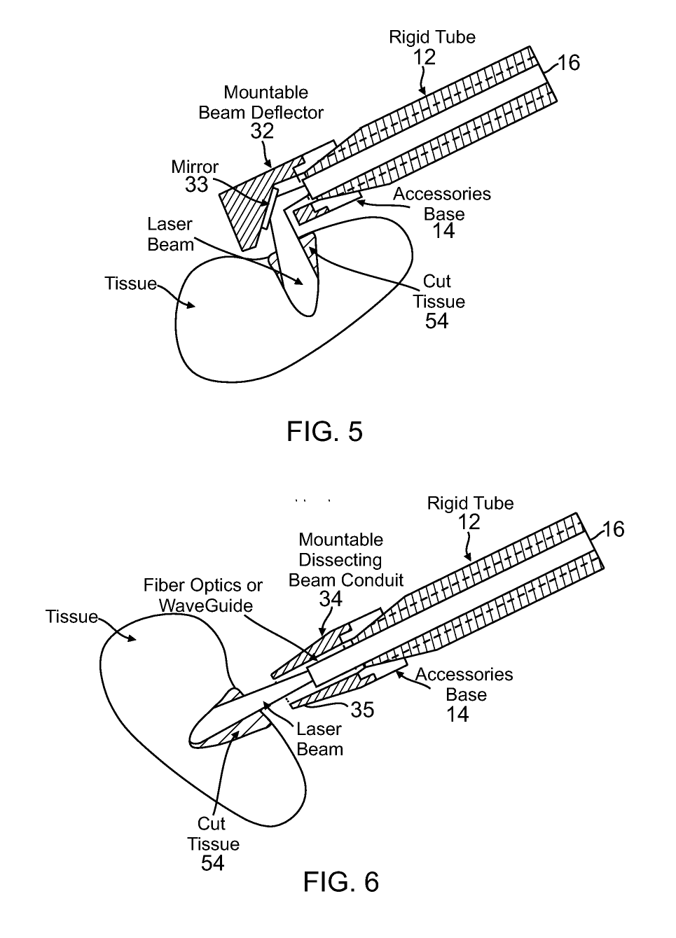

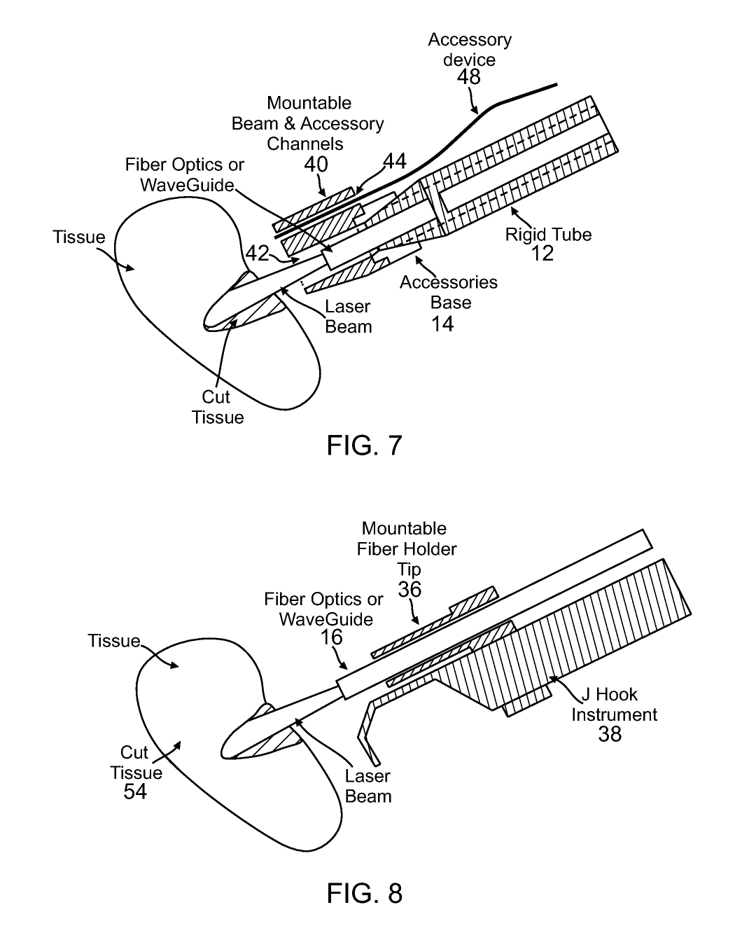

Referring to FIG. 5, in another embodiment of the invention, an instrument or accessory according to the invention includes a beam deflector 32 having a reflective mirror 33 that is designed and configured to removably mount or connect to the accessories base 14. In one embodiment of the accessories base 14 according to the invention, the accessories base 14 is designed and configured as a separate mountable/connectable unit for mounting/connecting to the instrument 10. In another embodiment of the accessories base 14 according to the invention, the accessories base 14 is designed and configured integral with the instrument 10 such that the accessories base 14 and the instrument 10 are one unit onto which various instruments or accessories may be mounted/connected. The beam deflector 32 with reflective mirror 33 is constructed and arranged with a mounting or connection device and configuration for mounting/connecting to and removing from the accessories base 14. The beam deflector 32 with mirror 33 may be also constructed and arranged to reflect a laser beam emitted from the tip of the instrument 10 in a direction or at an orientation that is substantially perpendicular relative to the axis of the beam emitted from wave guide 16, as shown in FIG. 5. The beam deflector 32 with mirror 33 may be positioned or oriented to deflect a laser beam at any of a range of other predetermined or defined angles. As shown in FIG. 5, the deflector 32 with mirror 33 may be positioned at any preferred or selected angle. Accumulation of tissue debris on the mirror should be avoided and various mechanisms may be employed for such purpose. One example of such a mechanism to prevent or to minimize the accumulation of debris on the mirror 33 is an accessory channel, such as accessory channel 44 shown in and discussed below with reference to FIG. 7, defining a hollow conduit through which a fluid, such as a saline solution, may be introduced to impinge upon mirror 32 and wash away any debris on mirror 33. The beam deflector 32 with mirror 33 is further designed and configured with a profile and size such that it will not interfere in passing the instrument 10 through a trocar 22 or other device that enables such passage when the beam deflector with mirror 33 is mounted on the accessories base 14.

Referring to FIG. 6, in another embodiment of the invention, an instrument or accessory according to the invention includes a dissection device 34 that is designed and configured to removably mount or connect to the accessories base 14. In one embodiment of the accessories base 14 according to the invention, the accessories base 14 is designed and configured as a separate mountable/connectable unit for mounting/connecting to the instrument 10. In another embodiment of the accessories base 14 according to the invention, the accessories base 14 is designed and configured integral with the instrument 10 such that the accessories base 14 and the instrument 10 are one unit onto which various instruments or accessories may be mounted/connected. The dissection device 34 is constructed and arranged with a mounting or connection device and configuration for mounting/connecting to and removing from the accessories base 14. The dissection device 34 may perform mechanical tissue dissection in conjunction with tissue treatment of a laser beam. In this embodiment of the invention, the dissecting device 34 is further constructed and arranged as a tube-like tip through which the laser wave guide 16 passes, but is retracted inwardly a specified minimum distance from the tip 35 and within the dissection device 34, such that the dissecting device 34 can be used to perform mechanical dissection without subjecting the wave guide 16 to damage resulting from the intentional or unintentional poking of the instrument into tissue or damage from tissue debris. The dissecting device 34 is further designed and configured of a size and profile such that it will not interfere in passing the instrument 10 through a trocar 22 or other device that enables such passage when the dissecting device 34 is mounted on the accessories base 14.

Referring to FIG. 7, in another embodiment of the invention, an instrument or accessory according to the invention includes a multi-channel tip device 40 that is designed and configured to removably mount or connect to the accessories base 14. The accessories base 14 according to one embodiment of the invention may be integral with the instrument 10 along the distal end of the instrument 10. Alternatively, the accessories base 14 according to another embodiment of the invention may define a mountable/connectable unit that is mounted/connected to the instrument 10 along the distal end of the instrument 10. The multi-channel tip device 40 is constructed and arranged with a mounting or connection device and configuration for mounting/connecting to and removing from the accessories base 14. The multi-channel tip device 40 is further constructed and arranged to define multiple channels, e.g., two channels, substantially within its interior including, in one embodiment of the device 40 according to the invention, an optical fiber(s) or wave guide channel 42 and an accessory channel 44. The channel 42 facilitates passing of optical fiber(s) or a wave guide 16 through the instrument 10 in such a manner to enable the fiber(s) or wave guide 16 to deliver laser energy to the target tissue 54. The accessory channel 44 may be designed and configured to accept and to facilitate the positioning and/or passing of any of a variety of other instruments or accessories 48. For example, in some embodiments of the device 40 according to the invention, accessories 48 that the device 40 helps to position and/or pass to help to treat the target tissue 54 include, but are not limited to, other energy types (radio frequency and ultrasound energy), or a vacuum, or a fluid, or other media that help to treat the target tissue 54. In other embodiments of the device 40 according to the invention, instruments 48 that the device 40 helps to position and/or pass to help to treat the target tissue 54, or some function(s) related to such treatment, include, but are not limited to, a sensing device or sensor, e.g., a temperature measuring sensor, tissue analyzer, illumination fiber(s) and a vision sensing device or sensor, e.g., a CCD or CMOS. Such instruments may be used to monitor the effects of the tissue treatment and to close a treatment loop, for example, by discontinuing delivery of laser energy from the instrument 10 to the target tissue 54.

Referring to FIG. 8, in a further embodiment of the invention, an instrument or accessory according to the invention includes a wave guide or fiber channel holder 36 that is designed and configured to removably mount or connect to various types of surgical instruments, e.g., j-hook, grasper or scissors 38, and to contain the wave guide 16 or optical fiber (s) for delivery of laser energy to target tissue 54. The holder 36 is constructed and arranged to facilitate passage of the fiber(s) or wave guide 16 such that laser energy may be emitted to the target tissue 54. The holder 36 is also constructed and arranged to receive or accept and to thereby mount or connect one or more instruments or accessories including, but not limited to, the standoff 26, the backstop 28, the beam deflector 30, the beam deflector 32 with mirror 33, and the dissecting device 34, as described above with reference to FIGS. 2-6, to the instrument 10. For example, in the instance that the beam deflector 32 with mirror 33 of FIG. 5 is mounted on the distal end of the accessories base, in place of the j-hook instrument 38 shown in FIG. 8, a hollow conduit to carry a fluid to impinge on mirror 33 may be mountable to clear debris from the mirror, as discussed in connection with FIG. 5. In another example, as shown in FIG. 8, in one embodiment, holder 36 according to the invention is mounted to a j-hook such that the surgical instrument may perform its function as part of the tissue treatment and the wave guide 16 or optical fiber(s) may deliver laser energy to the target tissue 54. In this manner, the holder 36, which is mounted/connected to some surgical instrument, enables delivery of laser energy to the target tissue 54 in addition to the functionality of the instrument it is connected to, thereby enabling a surgeon to employ the most suitable treatment during a procedure or operation without the need to interchange the instrument.

The instruments or accessories described above with reference to FIGS. 1-8 provide illustrative examples of a wide variety of instruments or accessories that the accessories base 14 may receive or accept for use with the tissue treatment instrument 10. The invention is not limited in this respect and envisions other instruments or accessories may be used in conjunction with the accessories base 14 and the instrument 10.

Having thus described at least one illustrative aspect of the invention, various alterations, modifications and improvements will readily occur to those skilled in the art. Such alterations, modifications and improvements are intended to be within the scope and spirit of the invention. Accordingly, the foregoing description is by way of example only and is not intended as limiting. The invention's limit is defined only in the following claims and the equivalents thereto.

1-11. (canceled)

12. An apparatus comprising:
a support structure, an accessories base on the distal end of the support structure, a treatment tip with a distal end and a proximal end, and a flexible waveguide coupled to the supporting structure, and further coupled to the treatment tip, the flexible waveguide being coupleable to a laser source generating laser radiation and configured to deliver the laser radiation and to emit a beam of the generated laser radiation from a distal end of the flexible waveguide to irradiate a target tissue of a patient,
wherein the treatment tip is mounted at its proximal end on one of a removably mounted or permanently mounted accessories base on the distal end of the support structures.

13. The apparatus of claim 12, wherein the treatment tip is a standoff mounted on the distal end of the accessories base that extends a distance from the distal end and terminates a selected distance from the distal end.

14. The apparatus of claim 12, wherein the treatment tip is a backstop mounted on the distal end of the accessories base that extends a distance from the distal end and terminates a selected distance from the distal end.

15. The apparatus of claim 12, wherein the treatment tip is a beam deflector mounted on the distal end of the accessories base, the beam deflector having a passage for receiving the flexible waveguide and bending the waveguide a selected amount from the longitudinal axis of the flexible waveguide.

16. The apparatus of claim 12, wherein the treatment tip is a beam deflector mounted on the distal end of the accessories base, the beam deflector comprising a housing having a reflector disposed at an angle with respect to the beam of laser radiation from the flexible waveguide to deflect the beam of laser radiation impinging on the reflector and redirect the beam to a target tissue of a patient.

17. The apparatus of claim 16, wherein the reflector is disposed at an angle to deflect the beam approximately 90°.

18. The apparatus of claim 16, wherein the reflector is disposed at an angle to deflect the beam approximately in the range of 1° to 90°.

19. The apparatus of claim 16, wherein the reflector is a mirror.

20. The apparatus of claim 16, further comprising a conduit associated with the support structure, the conduit being capable of directing a fluid at the reflector to clear debris from the reflector.

21. The apparatus of claim 12, wherein the treatment tip is a dissector mounted on the distal end of the accessories base, the dissector enclosing the distal end of the flexible waveguide, the waveguide being located spaced back from the distal end of the treatment tip to substantially eliminate contact of the distal end of the flexible waveguide and the target tissue of a patient.

22. The apparatus of claim 12, wherein the treatment tip comprises at least two lumens mounted on the distal end of the accessories base, one of the at least two lumens containing the flexible waveguide, the other of the at least two lumens containing one of more of: an optical fiber, a source of fluid, a temperature sensoring device, an illumination optical fiber, a vision sensing device, and a tissue analyzer.

23. The apparatus of claim 12, wherein the treatment tip further includes at least one further mounting, the at least one further mounting being adapted to support at least one of: a standoff, a j-hook, a backstop, a beam deflector, a beam deflector with reflector, a dissecting device, an illuminating device, a sensor device, a viewing device, and a fluid carrying device.
